(19) United States
(12) Patent Application Publication
(10) Pub. No.: US 2025/0312078 A1
(43) Pub. Date: Oct. 9, 2025

(54) ORTHOPEDIC BALL TIP GUIDEWIRE POSITIONING DEVICE

(71) Applicant: BOARD OF REGENTS, THE UNIVERSITY OF TEXAS SYSTEM, Austin, TX (US)

(72) Inventor: Justin E. Bird, Houston, TX (US)

(21) Appl. No.: 18/866,377

(22) PCT Filed: May 18, 2023
(86) PCT No.: PCT/US2023/067165

§ 371 Date: Nov. 15, 2024

Related U.S. Application Data
(60) Provisional application No. 63/364,896, filed on May 18, 2022.

(51) Int. Cl. A61B 17/88; A61B 34/20; A61M 25/09

(52) U.S. Cl. CPC A61B 17/8897; A61B 34/20; A61M 2025/09008; A61M 2025/09166; A61M 2025/09175

(57) ABSTRACT

An orthopedic guidewire positioning device comprising: an elongated sheath extending between a proximal end and a distal end; the elongated sheath further comprising a guidewire channel extending through the elongated sheath to a guidewire opening at the distal end of the elongated sheath, the guidewire channel configured to receive a guidewire such that a distal end of the guidewire extends out of the guidewire opening at the distal end of the elongated sheath; an inflatable member proximate the distal end of the elongated sheath, the device configured to inflate the inflatable member such that the inflatable member extends away from a first side of the elongated sheath as it inflates to deflect the distal end of the elongated sheath in a direction opposite a direction along which the inflatable member extends.

### CROSS-REFERENCE TO RELATED APPLICATIONS

This application claims priority to and the benefit of U.S. Provisional Patent Application Ser. No. 63/364,896, filed May 18, 2022, the contents of which is incorporated herein by this reference as if fully set forth herein.

### FIELD OF THE DISCLOSURE

The field of this disclosure relates to a system for placement of intramedullary ball tip guidewires. Placement of such guidewires within a patient's bone can present navigation challenges. The present disclosure thus provides a system for more accurately navigating a ball tip guidewire through bone or between fractured fragments.

### BACKGROUND

Various types of bone fractures or breaks can require intramedullary instrumentation to be used during repair surgery and/or fracture management. During certain types of surgery, it can be necessary to insert a guide wire into a patient's bone. A reamer can then be placed over the guidewire in order to drill/prepare the bone to receive an implant. Many times, the guidewire will have a ball tip on its end. The ball tip is positioned to help prevent the reamer from overrunning past the end of the guidewire. For a fracture repair of a long bone, navigating the ball tip guidewire to the desired position is a relatively straightforward process. However, new technology has emerged that now enables intramedullary instrumentation and repair of smaller bones and more irregularly shaped bones, such as the forearm or the pelvis. When navigating a ball tip guidewire around a curve or other non-straight/non-linear bone fracture, the surgeon has limited control of the distal tip of the ball tip guidewire. The guidewire can easily stray and become malpositioned.

In order to address these navigation challenges, sometimes a surgeon will bend the ball tip guidewire near its tip area in order to help maneuver the guidewire around an angle. Exemplary ball tip guide wires and reamers are shown at FIGS. 5A and 5B. FIG. 5B shows a ball tip guidewire 16 having a bend 50 at or near its tip. This bend 50 is added by the surgeon in order to help navigate a curved or tortuous route in a bone (as opposed to navigating a straight long bone). FIG. 5B shows a reamer 52 advanced up to the bend 50, but unable to extend past the bend 50.

Although this bent tip may be helpful in navigating the guidewire, it does not always ensure adequate placement. For example, this does not give the surgeon the desired control of the distal end of the ball tip guidewire and can cause difficulty in placing an intramedullary nail. In general, surgeons have limited control over the distal tip of the guidewire, and the guidewire can stray and become mal-positioned. In some instances, the distal end of the guidewire can even pass through fractures or pathologic lesions in the bone entering the soft tissues, which is undesirable. Efforts to reposition the guidewire can ultimately cause damage to the bone and surrounding soft tissues, as well as increase surgical time. Reamers may also have a difficult time reaming past a surgeon-made bend, rendering the guidewire less effective. Improvements to navigating ball tip guidewires through a patient's bone are thus desirable.

### BRIEF SUMMARY

In use, the sheath is directed through the bone, to the surgical area. If the sheath reaches a difficult angle to be navigated, the inflatable side balloon can be inflated in order to push the sheath away from the inner edge of the bone and to allow the surgeon to steer the tip of the sheath inside the bone. The ball tip guidewire can then be inserted through the sheath (through a central channel of the sheath) into the correct position. The ball tip of the guidewire extends out and through the central channel of the sheath and can help guide other surgical instruments to the surgical area.

In certain examples, there is provided an orthopedic guidewire positioning device, comprising: a sheath comprising an internal diameter and a side wall channel; the internal diameter configured to receive a guidewire therethrough, the side wall channel configured to receive an inflatable side balloon; the side wall channel comprising a side opening in an external perimeter of the sheath, wherein the inflatable side balloon is configured to be positioned within the side wall channel and inflatable through and out the side opening.

In any of the above or subsequent examples, the inflatable balloon is positioned at the end of a fluid conduit, wherein the fluid conduit is positioned through the side wall channel.

In any of the above or subsequent examples, there is provided method for navigating a guidewire into a surgical area of an intramedullary space, comprising: providing the device of nay embodiments described herein; advancing the device to the surgical area; inflating the inflatable side balloon, causing the sheath to divert away from a navigational challenge; and continuing to advance the device to the surgical area.

### BRIEF DESCRIPTION OF THE DRAWINGS

FIG. 1 is a perspective view showing one embodiment of an orthopedic guidewire positioning device according to this disclosure.

FIG. 2 is a cross-sectional view of the device of FIG. 1 at the opening from which an inflatable side balloon extends.

FIG. 3 is a side plan view of the device of FIG. 1.

FIG. 4 is a close up view of the distal end of the sheath with a guidewire extending therethrough and an inflatable side balloon inflated.

FIG. 5A shows a side view of a bent guidewire tip.

FIG. 5B shows the bent guidewire tip of FIG. 5A with a reamer advanced thereto.

### DETAILED DESCRIPTION

The described embodiments provide an orthopedic guidewire positioning device 10. The device 10 generally includes an elongated sheath 12 with an open central channel that defines an internal diameter 14 that can receive a guidewire 16 in use. (In most instances, the guidewire 16 will generally be a ball tip guidewire that has a rounded ball 18 at its distal end 20.) The sheath 12 is provided with a separate side wall channel 22. The side wall channel 22 is separate and independent from the central channel that defines the internal diameter 14 of the sheath 12. The side wall channel 22 is sized and dimensioned to receive a fluid conduit 24 that has an inflatable side balloon 28 positioned at the end thereof. In one example, this may be a balloon catheter. The side wall channel 22 of the sheath 12 defines an side opening 26 at the distal end 30 of the sheath. This opening is generally provided as a cut out part/window in the external perimeter 25 of the sheath 12. The inflatable side balloon 28 can be deployed through the side opening 26 in use. The presence of the side opening 26 allows the inflatable side balloon 28 to push out of the side opening 26 upon inflation, which will be described in more detail below.

In use, the fluid conduit 24 is a tubing or balloon catheter that extends through the interior of the side wall channel 22 of the sheath. The inflatable side balloon 28 is typically secured at the distal end of the fluid conduit 24. The body of the fluid conduit 24 can be positioned within the side wall channel 22 of the sheath 12 in use. The proximal end of the fluid conduit 24 (the part of the fluid conduit that does not extend through the sheath) extends outside the patient and has an attachment area for a syringe or other inflation element 32. When the syringe or other inflation element 32 is activated (typically squeezed), this delivers fluid (either air or saline) to the inflatable side balloon 28 to cause the balloon to inflate. This may be a manual activation, such as squeezing, or inflation may be operated via a foot pedal, or any other appropriate option.

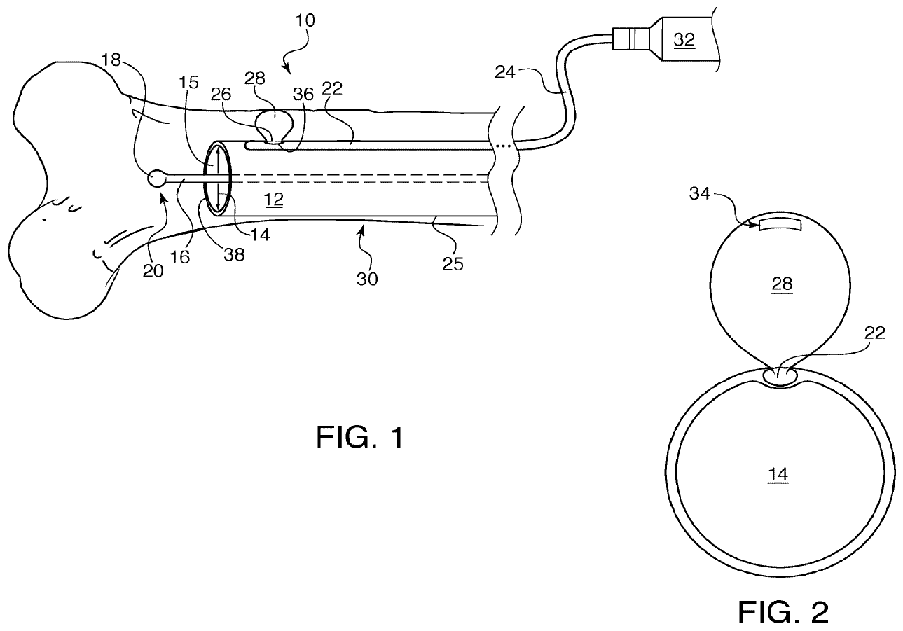

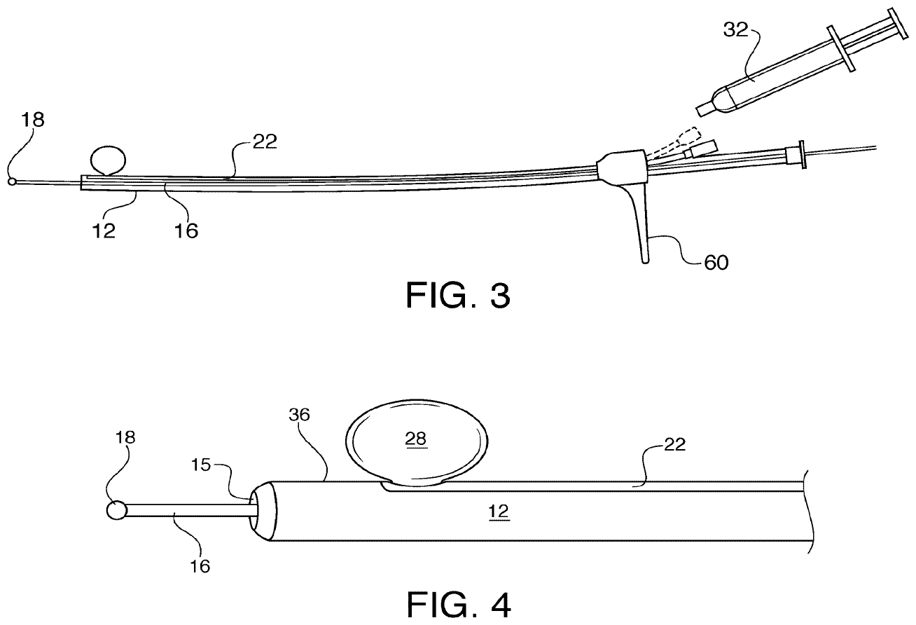

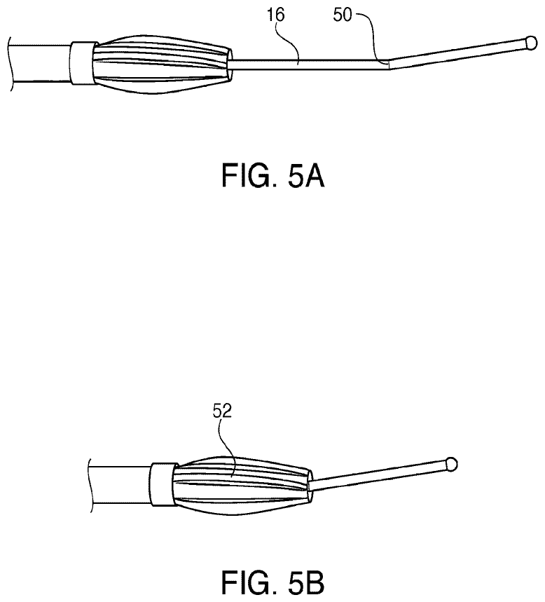

Because the side opening 26 is at the distal end 30 of the sheath 12 and because the side opening 26 is along a side wall 36 of the external perimeter 25 of the sheath, upon inflation, the inflatable side balloon 28 extends out from a side of the sheath 12 as shown by FIG. 1. Inflating the inflatable side balloon 28 pushes the sheath 12 away from an inner edge of the bone (or whatever surface the sheath tip 38 is bumping up against), allowing the surgeon to navigate a curve more easily and to more effectively steer the tip 38 of the sheath. Rather than bumping up against a bone side wall or extending through soft tissues, the sheath 12 can be deflected away from its current course due to inflation of the inflatable side balloon 28. The inflatable side balloon 28 pushes the side wall 36 of the sheath 12 away from whatever the inflatable side balloon 28 pushes against, causing the tip 38 of the sheath 12 to curve or otherwise deflect away from its current course.

Either prior to insertion and placement of the sheath 12 or during surgery itself, the fluid conduit 24 may be threaded or otherwise inserted into the side wall channel 22 so that the inflatable side balloon 28 is generally aligned with the side opening 26 near the distal end 30 of the sheath 12. It is possible for the sheath 12 and the fluid conduit 24/inflatable side balloon 28 to be inserted and navigated together. Alternatively, it is possible for the sheath 12 to be inserted and if more detailed navigation is required, the fluid conduit 24/inflatable side balloon 28 can be subsequently inserted through the side wall channel 22. Alternatively, the balloon may be permanently mounted at the side opening 26, and the fluid conduit 24 may simply be provided by the side channel 22 itself. Delivering saline or air or other inflation fluid directly to the side channel 22 can cause the inflatable side balloon to inflate.

During surgery, the sheath 12 is generally navigated to the intramedullary location of interest. If needed, the inflatable side balloon 28 can be inflated to extend out of the side opening 26 in order to push the side wall 36 of the sheath 12 away from a navigational challenge. Once appropriate navigation is achieved, the ball tip guidewire 16 can then be inserted through the internal diameter 14 of the sheath 12 and can extend out from the diameter opening 15 at the tip 38 of the sheath. Once the ball-tipped guidewire is in the desired position, the balloon is deflated and the sheath removed, leaving the guidewire in position. Surgery may continue as per the surgical plan.

In some examples, it is possible for the inflatable side balloon 28 to be provided with a radiopaque marker 34 which can help the surgeon see the location of the balloon 28 under fluoroscopy or x-ray or other radiograph. This feature may assist the surgeon in the initial positioning of the sheath 12 simply because the location of the inflatable side balloon 28 with respect to the tip 38 of the sheath is a known distance. This feature may assist the surgeon in navigation of the sheath 12 because inflation of the balloon can be visualized.

In some examples, it is possible for the sheath tip 38 to be provided with a radiopaque marker. This can similarly help the surgeon see the location of the tip 38 under fluoroscopy or x-ray or other radiograph.

It is possible for there to be provided a working handle 60 at the proximal end of the sheath 12 (the end that is positioned outside the patient). In this option, the working handle 60 can be positioned or otherwise marked so that its location with respect to the inflatable side balloon 28 remains constant. If the handle is rotated 90°, then the surgeon knows that the balloon is also rotated 90°. Additionally or alternatively, there may be provided a marker on the sheath 12 and/or on the fluid conduit 24 itself (attached to the inflatable side balloon 28) or any other part of the system that extends outside the patient. The marker can allow the surgeon to know which side of the sheath 12 the balloon 28 is on, even when the sheath is rotated and turned during insertion, without using an x-ray or any other type of imaging. For example, the marker may be provided on the same side of the end of the sheath that the side opening 26 of the side wall channel 22 is on. In a specific example, if the marker is on the right side of the sheath, this can tell the surgeon that the side opening 26 is also on the right side of the sheath.

Side deployment of the balloon is important (vs. deployment from the forward tip of the sheath) because it is the side edge of the sheath that needs to be pushed away from the side of the bone in order to make the turn. This eccentricity of having an inflatable balloon 28 that can extend from only one side of the sheath can greatly increase navigation and usability of a guidewire system that needs to be positioned within a patient's bone. The tip 38 of the sheath 12 can only be pushed in one direction, away from where the balloon inflates. The directional force created by the inflation moves the sheath.

The material of the sheath 12 may be somewhat malleable surgical plastic. Non-limiting examples include thermoplastic polyurethane, polypropylene, polythylene, or any other appropriate material. The material of the inflatable balloon 28 may be similar to balloons used for balloon kyphoplasty or any other type of inflatable catheter balloon. Non-limiting examples include polyesters, PEBA, polyurethane, silicone, or any other appropriate material. It is generally envisioned that the material of the inflatable balloon be able to withstand pressures up to about 700 psi for best results. The strength profile of the balloon should be such that it can extend and press against a bone with sufficient force to move the sheath tip without bursting.

The subject matter of certain embodiments of this disclosure is described with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It should be understood that different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

1. An orthopedic guidewire positioning device comprising:
(a) an elongated sheath extending between a proximal end and a distal end;
(b) the elongated sheath further comprising a guidewire channel extending through the elongated sheath to a guidewire opening at the distal end of the elongated sheath, the guidewire channel configured to receive a guidewire such that a distal end of the guidewire extends out of the guidewire opening at the distal end of the elongated sheath;
(c) an inflatable member proximate the distal end of the elongated sheath, the device configured to inflate the inflatable member such that the inflatable member extends away from a first side of the elongated sheath as it inflates to deflect the distal end of the elongated sheath in a direction opposite a direction along which the inflatable member extends.

2. The orthopedic guidewire positioning device of claim 1, further comprising an inflation element configured to deliver fluid to the inflatable member, wherein actuation of the inflation element inflates the inflatable member only on the first side of the elongated sheath.

3. The orthopedic guidewire positioning device of claim 2, wherein the inflatable member is connected to a fluid conduit extending from the inflatable member towards the proximal end of the elongated sheath.

4. The orthopedic guidewire positioning device of claim 3, wherein the fluid conduit is separate from the guidewire channel.

5. The orthopedic guidewire positioning device of claim 4, wherein the fluid conduit extends through a side wall channel of the elongated sheath, the side wall channel being a separate channel from the guidewire channel.

6. The orthopedic guidewire positioning device of claim 3, wherein the fluid conduit is removably installed in the side wall channel.

7. The orthopedic device of claim 2, wherein the inflatable member extends through a sidewall opening of the elongated sheath.

8. The orthopedic device of claim 7, wherein the device is configured to inflate the inflatable member such that the inflatable member pushes out of the sidewall opening of the elongated sheath.

9. The orthopedic device of claim 2, wherein the inflatable member comprises a radiopaque marker.

10. The orthopedic device of claim 2, wherein the distal end of the sheath comprises a radiopaque marker.

11. The orthopedic device of claim 2, further comprising a working handle connected to the sheath, wherein rotation of the working handle rotates the elongated sheath and the inflatable member.

12. The orthopedic device of claim 2, wherein the guidewire is a ball tip guidewire.

13. The orthopedic device of claim 2, wherein the guidewire is a reamer guide.

14. A method for navigating a guidewire in an intramedullary space, the method comprising:
(a) inserting an elongated sheath into an intramedullary space along an insertion pathway, the elongated sheath extending between a proximal end and a distal end and comprising a guidewire channel extending through the elongated sheath and configured to receive a guidewire;
(b) inflating an inflatable member proximate the distal end of the elongated sheath, wherein inflation of the inflatable member deflects the distal end of the elongated sheath relative to the insertion pathway;
(c) after deflecting the distal end of the elongated sheath, further inserting the elongated sheath into the intramedullary space.

15. The method of claim 14, further comprising using the elongated sheath to position a guidewire in the intramedullary space.

16. The method of claim 15, further comprising installing a reamer over the guidewire.

17. The method of claim 14, wherein the elongated sheath is connected to a working handle, wherein the method further comprises rotating the working handle to rotate the sheath and the inflatable member about the insertion pathway.

18. The method of claim 14, wherein inflating the inflatable member comprises inflating the inflatable member such that the inflatable member extends away from a first side of the elongated sheath as it inflates to deflect the distal end of the elongated sheath away from the first side of the elongated sheath.

19. The method of claim 18, further comprising actuating an inflation element proximate the proximal end of the elongated sheath to inflate the inflatable member only on the first side of the elongated sheath.

20. The method of claim 14, wherein inflating the inflatable member deflects the distal end of the elongated sheath away from an obstruction in the intramedullary space.
